(19) United States
(12) Patent Application Publication
(10) Pub. No.: US 2026/0102174 A1
(43) Pub. Date: Apr. 16, 2026

(54) FOREIGN BODY RETRIEVAL DEVICE FOR AN ENDOSCOPIC DEVICE, ENDOSCOPIC DEVICE AND METHOD FOR MANUFACTURING A FOREIGN BODY RETRIEVAL DEVICE

(71) Applicant: FUJIFILM CORPORATION, Tokyo (JP)

(72) Inventor: Anton TSYRULNYKOV, Nurenberg (DE)

(21) Appl. No.: 19/350,985

(22) Filed: Oct. 6, 2025

(30) Foreign Application Priority Data
Oct. 10, 2024 (EP) 24 205 733.9

(51) Int. Cl. A61B 17/221; A61B 17/00; A61L 31/04

(52) U.S. Cl. CPC A61B 17/221; A61L 31/048; A61B 2017/00358; A61B 2017/00526; A61B 2017/00818; A61B 2017/00955; A61B 2017/2215

(57) ABSTRACT

A foreign body retrieval device for an endoscopic device, including a retrieval section, that forms a retrieval net, and an adjacent fastening section that is provided for fastening the foreign body retrieval device in the endoscopic device. At least the retrieval net of the retrieval section and the fastening section are in one piece as a textile structure which is produced by interlinking at least one filament. At the fastening section the structure forms at least one mounting loop, at which the foreign body retrieval device is to be fastened in the endoscopic device.

### CROSS-REFERENCE TO RELATED APPLICATIONS

The present application claims priority of EP 24 205 733.9, filed Oct. 10, 2024, the priority of this application is hereby claimed, and this application is incorporated herein by reference.

### BACKGROUND OF THE INVENTION

The invention relates to a foreign body retrieval device for an endoscopic device, comprising a retrieval section, that forms a retrieval net, and a fastening section adjacent thereto, which is provided for fastening the foreign body retrieval device in the endoscopic device, wherein at least the retrieval net of the retrieval section and the fastening section are in one piece as a textile structure which is produced by interlinking at least one filament. Furthermore, the invention relates to an endoscopic device and a method for manufacturing a foreign body retrieval device.

Foreign body retrieval devices are used in endoscopy, for example, to remove foreign bodies from the gastrointestinal tract. The foreign body to be removed may be an object swallowed by the patient or a medical device such as a stent, probe, etc. In addition, polyps that have been removed are also retrieved using endoscopic foreign body retrieval devices. In addition to foreign body forceps and grippers, foreign body retrieval devices with retrieval nets are also used for this purpose. Retrieval nets have the advantage that, in addition to simple, intuitive handling, they also allow the retrieval object to be securely enclosed during gastroesophageal passage with minimal risk of aspiration in the event of loss. Retrieval nets are mostly used in gastroscopes or enteroscopes, which can also be very thin for children.

A foreign body retrieval device with a retrieval net is usually fully retracted into a tube, when the endoscope is inserted, and is pushed out via a handpiece to retrieve the foreign body. In some cases, the retrieval net is attached to a loop formed by a wire-shaped element, whereby the loop expands, preferably in a circular, elliptical, or oval shape, when a pull cord or wire of the endoscope is pushed out, causing the net to be stretched. Thereby, it must always be ensured that the retrieval net is reliably attached to the endoscopic device.

U.S. Pat. No. 11,871,957 B2 describes a foreign body retrieval device that has a retrieval section and an adjacent fastening section. While the leaf-like retrieval section forms a retrieval net for the foreign body retrieval device, the fastening section is used to fasten the foreign body retrieval device in an endoscopic device. For this purpose, the fastening section has a rectangular shape, wherein the fastening section is wrapped around the corresponding connection point in the endoscopic device and tied tightly when fastening the foreign body retrieval device. The retrieval section and the fastening section are made in one piece from filament, which is intertwined to form meshes, thereby creating a textile structure that forms the retrieval section and the fastening section.

### SUMMARY OF THE INVENTION

Starting from the prior art described above, the present invention aims to create a foreign body retrieval device that is characterized by the lowest possible manufacturing costs and at the same time allows reliable attachment within an endoscopic device.

According to invention, a foreign body retrieval device comprises a retrieval section, which forms a retrieval net, and an adjacent fastening section, which is provided for fastening the foreign body retrieval device in an endoscopic device. At least the retrieval net of the retrieval section and the fastening section are in one piece as a textile structure, which is produced by interlinking at least one filament.

The foreign body retrieval device according to the invention thus has a retrieval section and a fastening section, wherein the retrieval section and the fastening section are adjacent to each other, i.e., they are designed to be immediately neighboring each other. The retrieval section of the foreign body retrieval device defines a retrieval net which, when the foreign body retrieval device is used in an endoscopic device, serves to receive a foreign body to be retrieved. Preferably, the retrieval net has a bulbous shape in a stretched state, which the retrieval net has when the foreign body retrieval device is used to retrieve a foreign body. This creates a volume in the retrieval net for receiving the foreign body to be retrieved, which simplifies the reception and enclosing of the foreign body for an operator of the endoscopic device when the foreign body retrieval device is used. In particular, the retrieval section has a leaf-like shape, which is preferably defined by an outer contour of the retrieval section. This creates a structure of the retrieval section which, as an alternative but preferably in addition to the bulbous shape of the retrieval net, simplifies the handling of the foreign body retrieval device when used in an endoscopic device.

The fastening section is provided in the foreign body retrieval device according to the invention for fastening the foreign body retrieval device in the endoscopic device. In this respect, when the foreign body retrieval device is used in an endoscopic device, the foreign body retrieval device is fastened to the rest of the endoscopic device at the fastening section, and subsequently, actuating movements are also initiated at the fastening section in the foreign body retrieval device. It is particularly preferred that the fastening section serves as the sole fastening of the foreign body retrieval device in the endoscopic device.

In the present case, the fastening section and at least the retrieval net of the retrieval section are formed together in one piece, wherein this one-piece design is achieved by at least the retrieval net of the retrieval section and the fastening section being made by a textile structure. This textile structure is made from at least one filament, which is interlinked and thereby defines the textile structure. In this respect, a “textile structure” is to be understood as a flat or three-dimensional structure which has been realized by interlinking one or more filaments. In the sense of the invention, a “filament” is to be understood as a fiber with a preferably practically unlimited length, so that the filament is then in particular a continuous fiber. In the sense of the invention, the term “to interlink” also includes the knotting of the at least one filament.

Within the scope of the invention, the textile structure could be formed by intertwining one or more filaments to form meshes. In this case, the textile structure consists of meshes, each of which is formed by intertwining a loop formed by the at least one filament with another loop formed by the at least one filament. Accordingly, the textile structure in this variant of the invention could also be referred to as a mesh fabric. The meshes of the textile structure could be formed by knitting a single filament or several filaments, so that in this case the textile structure is a knitted fabric. Alternatively, the textile structure could also be produced as a knitted textile, for example as a weft-knitted fabric from a single filament or as a warp-knitted fabric from several filaments. In this way, a suitable textile structure can be produced for forming the fastening section and at least the retrieval net of the retrieval section.

Alternatively, the textile structure could also be realized as a fabric by weaving several filaments or by braiding several filaments. This is because this also allows a suitable structure of a textile structure to be realized for the fastening section and at least the retrieval net of the retrieval section. As a further alternative, the textile structure could also be formed by knotting one or more filaments, for example, similar to macramé, platting, or crocheting.

According to the invention, the invention now comprises the technical teaching that at the fastening section the structure forms at least one mounting loop at which the foreign body retrieval device is to be fastened in the endoscopic device. In other words, at the fastening section of the foreign body retrieval device of the invention the textile structure defines at least one mounting loop at which fastening of the foreign body retrieval device in the endoscopic device is to be carried out.

This design of a foreign body retrieval device has the advantage that the forming of at least one mounting loop allows a form-fitting attachment to the fastening section of the foreign body retrieval device, which also enables a reliable and resilient attachment of the foreign body retrieval device according to the invention in the endoscopic device. Due to the one-piece design of at least the retrieval net and the fastening section by means of the textile structure, which also forms the at least one mounting loop of the fastening section, the manufacturing costs of the foreign body retrieval device according to the invention can also be kept low. This is because the number of necessary assembly steps can be reduced during manufacture, wherein the interlink of the at least one filament can also be automated in a simple manner in the sense of a continuous manufacturing process.

In addition, the foreign body retrieval device can be made highly flexible by designing at least part of it from the textile structure, making it easier to bend and thus easier to navigate through complex paths when used in an endoscopic device. This also reduces the risk of injury to the patient, wherein the at least one filament can additionally be made of a soft material in order to further minimize the risk of injury or to increase treatment comfort. With a suitable choice of material for the at least one filament, biocompatibility can also be achieved, thereby reducing the risk of rejection reactions or inflammation and/or reducing the weight of the foreign body retrieval device and/or increasing the ergonomics when using the foreign body retrieval device.

Depending on the material, reliable sterilization is also possible and a long service life of the foreign body retrieval device can be achieved. In addition, depending on the respective material of the at least one filament, certain properties of the foreign body retrieval device can be realized, such as a certain elasticity, a certain stiffness, and/or a certain conductivity, wherein these properties can also be specifically designed to vary in different areas when different materials are combined.

Furthermore, complex designs for the foreign body retrieval device can also be easily realized using the textile structure. In addition, sensors or actuators can be embedded in the textile structure at suitable locations with little effort.

In the context of the invention, a “mounting loop” refers to a ring-like area formed on the fastening section and through which a passage is circumscribed. In this way, the at least one filament can also form the at least one ring-like mounting loop by crossing over in at least one area. A fastening element that is provided on the side of the remaining endoscopic device, for example a hook or similar, can be passed through the circumscribed passage in order to create a form-fitting connection between the fastening section of the foreign body retrieval device and the remaining endoscopic device.

According to one embodiment of the invention, in the retrieval section the retrieval net is surrounded by a support frame. This support frame forms, in particular, a load-bearing structure of the retrieval section, wherein this support frame preferably serves as a boundary for the retrieval net. It is further preferred that the retrieval net is stretched via the support frame when the foreign body retrieval device according to the invention is pushed out of the endoscopic device in its installed state with its retrieval section, in particular from a tube of the endoscopic device, in order to be able to receive an object to be retrieved in the stretched state of the retrieval net. It is preferred that the support frame is designed to be completely or almost completely circumferential to the retrieval net, wherein the support frame can be formed from one or more wire-like elements within the scope of the invention. The support frame can thus be in the form of a wire loop.

However, it is particularly preferred that the support frame is designed in one piece together with the retrieval net and the fastening section as the textile structure and has a higher rigidity compared to the retrieval net. This further reduces the manufacturing costs of the foreign body retrieval device according to the invention, as the support frame of the retrieval section is also formed by the textile structure in addition to the retrieval net and the fastening section. At the same time, the higher rigidity of the support frame compared to the retrieval net allows a suitable structure to be created for supporting the retrieval net and for tensioning it.

In a further development of the aforementioned embodiment, the higher rigidity of the support frame compared to the retrieval net is achieved by forming the support frame with a higher density than the retrieval net. The higher rigidity can be achieved within the scope of the invention by the textile structure forming the support frame with a higher density, i.e., a higher concentration of filaments, compared to the retrieval net. If the textile structure is knitted, the support frame could be formed in a garter stitch pattern, while the retrieval net is made in a stockinette stitch pattern. Alternatively or in addition, the higher stiffness can also be achieved by forming the textile structure from several filaments and using filaments with a higher density in the manufacture of the textile structure on the support frame compared to the manufacture of the textile structure on the retrieval net. Furthermore, as an alternative or supplement, the higher rigidity of the support frame could also be achieved by forming the textile structure from several filaments and using a filament made of a material with a higher strength to form the support frame.

Preferably, the support frame also forms a connection to the fastening section. This advantageously provides a suitable, resilient connection between the retrieval section and the adjacent fastening section.

According to one embodiment of the invention, the fastening section has a higher rigidity than the retrieval net. This allows the foreign body retrieval device according to the invention to have a resilient fastening section, via which linear actuating movements can also be transmitted to the retrieval section.

The higher rigidity of the fastening section compared to the retrieval net is preferably achieved by forming the fastening section with a higher density in comparison to the retrieval net. The textile structure can form the fastening section with a higher density, i.e., a higher concentration of filaments. If the textile structure is designed as a knitted fabric, the fastening section is preferably realized in a garter stitch pattern, while the retrieval net is manufactured in a stockinette stitch pattern in particular. Alternatively or in addition to this, the higher stiffness of the fastening section could also be achieved by forming the textile structure from several filaments and using filaments with a higher density in the manufacture of the textile structure at the fastening section compared to the manufacture of the textile structure at the retrieval net. Furthermore, as an alternative or supplement, when manufacturing the textile structure from several filaments, a filament made of a material with a higher strength may also have been used to form the fastening section.

Another embodiment of the invention is that the fastening section is connected to the retrieval section by an elongated connecting part, which connects the retrieval section to the at least one mounting loop. This easily allows attachment of the foreign body retrieval device apart from the retrieval section and the retrieval net formed therein. In a further development of this embodiment, the connecting part can also form an actuating element of the endoscopic device, wherein the at least one mounting loop is provided for attachment to a handpiece of the endoscopic device. In this case, the elongated connecting part would then be designed with such an extension that the connection to the handpiece of the endoscopic device can be made directly via the connecting part, and as a result, an intermediate, separate actuating element, such as an actuating wire or cable, can be omitted.

In a further embodiment of the invention, the at least one filament is designed as a monocomponent, i.e., it consists of only one material. Preferably, the at least one filament is made of a thermoplastic material, wherein polyvinyl chloride or polyethylene are particularly preferred.

According to one embodiment of the invention, the textile structure forms exactly one mounting loop at the fastening section. This allows the foreign body retrieval device according to the invention to be fastened in a compact space.

Alternatively, the textile structure forms several mounting loops in the fastening section, wherein preferably exactly two mounting loops or exactly three mounting loops are formed. It is particularly preferred that the several mounting loops follow each other one after the other in a row, i.e., the mounting loops are formed in a row by the textile structure. The formation of several mounting loops has the advantage that the foreign body retrieval device according to the invention can be fastened in the endoscopic device by a thread-through of a tension element of the endoscopic device. This means that no kinking of the tension element is necessary. Furthermore, this increases the security of the mounting and improves the stability of the fastening, wherein also a press-fitting of the connection is simplified.

The invention also relates to an endoscopic device comprising a flexible tube. This tube has at least one lumen running in its longitudinal direction, through which an actuating element runs with radial play. A foreign body retrieval device according to one or more of the aforementioned embodiments is connected to this actuating element. The use of the foreign body retrieval device according to the invention in the endoscopic device has the advantage that the endoscopic device can thus be realized with low manufacturing costs and with reliable attachment of the foreign body retrieval device.

The foreign body retrieval device can be moved via the actuating element preferably between a position in which it is completely retracted into the tube and a position in which at least the retrieval section of the foreign body retrieval device is predominantly extended out of the tube. In the retracted position, the retrieval net of the foreign body retrieval device is folded up in the tube, while in the extended position it is preferably stretched out.

It is particularly preferable for the foreign body retrieval device according to the invention to be attached to the actuating element via the mounting loop of the fastening section, wherein the actuating element guided with play in the lumen is in particular an actuating wire or cable.

Alternatively, however, it is also a possible embodiment of the invention that the actuating element is formed by the connecting part of the foreign body retrieval device and the mounting loop of the foreign body retrieval device is attached to a handpiece. In this case, the fastening section of the foreign body retrieval device is equipped with the elongated connecting part, which then functions as an actuating element within the endoscopic device and extends from the retrieval section to the handpiece of the endoscopic device in order to fasten the handpiece via the mounting loop of the fastening section. This has the advantage of eliminating the need for a separate actuating element, as the fastening section of the foreign body retrieval device according to the invention also forms the actuating element.

Further according to the invention, in a method for manufacturing a foreign body retrieval device, a retrieval section forming a retrieval net and an adjacent fastening section provided for fastening the foreign body retrieval device in the endoscopic device are manufactured, wherein at least the retrieval net of the retrieval section and the fastening section are jointly designed as a one-piece textile structure which is formed by interlinking at least one filament.

In the context of the invention's method of manufacturing a foreign body retrieval device, a retrieval section and a fastening section are formed adjacent to each other, wherein the retrieval section and the fastening section are preferably formed immediately adjacent to each other. In addition, in the retrieval section, a retrieval net is provided, which is intended to receive a foreign body to be removed, such as a polyp.

In the invention's manufacturing process, the fastening section and at least the retrieval net of the retrieval section are formed together in one piece, wherein the fastening section and at least the retrieval net of the retrieval section are defined by a textile structure. This textile structure is made from at least one filament, which is interlinked to form the textile structure. Accordingly, a “textile structure” is understood to be a flat or three-dimensional structure that has been created by interlinking one or more filaments. In the sense of the invention, a “filament” is understood to be a fiber with a preferably practically unlimited length, so that the filament is then in particular a continuous fiber. In the sense of the invention, the term “to interlink” also includes the knotting of at least one filament.

Specifically, the textile structure could be formed by intertwining one or more filaments to form meshes. In this case, the textile structure is formed from meshes, each of which is created by intertwining a loop formed by the at least one filament with another loop formed by the at least one filament. Accordingly, the textile structure formed in this embodiment of the invention could also be referred to as a meshed fabric. The meshes of the textile structure could be formed by knitting a single filament or several filaments, so that in this case the textile structure is formed as a knitted fabric. Alternatively, the textile structure could also be produced as a knitted textile, for example as a weft-knitted fabric from a single filament or as a warp-knitted fabric from several filaments. This also results in a suitable textile structure for forming the fastening section and at least the retrieval net of the retrieval section.

Alternatively, the textile structure could also be produced as a fabric by weaving several filaments or by braiding several filaments. This also results in a suitable structure of a textile structure for realizing the fastening section and at least the retrieval net of the retrieval section. As a further alternative, the textile structure could also be formed by knotting one or more filaments, for example, similar to macramé, platting, or crocheting.

The invention now comprises the technical teaching that at least one mounting loop is formed by the structure at the fastening section, at which mounting loop the foreign body retrieval device is to be fastened in the endoscopic device. In other words, during the manufacture of the textile structure, at least one mounting loop is defined by the structure, wherein this at least one mounting loop serves to attach the foreign body retrieval device in the endoscopic device.

The invention's method of manufacturing a foreign body retrieval device has the advantage that it can be manufactured at low cost, as at least the retrieval net of the retrieval section and the fastening section are defined by interlinking the at least one filament during the manufacture of the textile structure. This is because the number of assembly steps required during production can be reduced, wherein the interlink of the at least one filament can also be easily automated. In addition, adjustments to different dimensions of the foreign body retrieval device can be easily implemented without the need for extensive changes to the machinery.

In a further embodiment of the invention, the foreign body retrieval device is designed in accordance with the method of the invention and in further accordance with one or more of the embodiments described above. This allows a suitable design of the foreign body retrieval device to be realized in each case.

Alternatively or in addition, a plurality of such foreign body retrieval devices are produced sequentially in one piece as a textile structure by interlinking at least one filament, wherein the foreign body retrieval devices are then separated from one another at separation points. This further reduces the manufacturing effort by enabling continuous production of foreign body retrieval devices. Foreign body retrieval devices can be manufactured in series by defining the retrieval section of the subsequent foreign body retrieval device following on from forming a fastening section of the preceding foreign body retrieval device using the textile structure, or vice versa. Alternatively or in addition to this, foreign body retrieval devices can also be manufactured side by side, for example by having the textile structure directly form the retrieval section of a neighboring foreign body retrieval device next to the retrieval section of one foreign body retrieval device. This allows for continuous production without any problems. Following the common production of several foreign body retrieval devices, they are then separated into individual foreign body retrieval devices by separation at the corresponding separation points.

In a further embodiment of the aforementioned embodiment, separation is carried out by means of hot cutting. This allows simple separation to be achieved and, in the case of using a thermoplastic material as filament, also by melting of the material prevents unwanted opening of the textile structure. Alternatively, the separation points of the foreign body retrieval devices can also be designed as predetermined breaking points, so that the foreign body retrieval devices can then be separated by bending at the predetermined breaking points.

The various features of novelty which characterize the invention are pointed out with particularity in the claims annexed to and forming a part of the disclosure. For a better understanding of the invention, its operating advantages, specific objects attained by its use, reference should be had to the drawings and descriptive matter in which there are illustrated and described preferred embodiments of the invention.

### BRIEF DESCRIPTION OF THE DRAWING

In the drawings:

FIG. 1 a view of a foreign body retrieval device according to a first embodiment of the invention;

FIG. 2 a perspective view of the foreign body retrieval device from FIG. 1;

FIG. 3 a sectional view of a part of an endoscopic device with the foreign body retrieval device according to FIGS. 1 and 2;

FIGS. 4 and 5 alternative designs of a fastening element of an endoscopic device for fastening the foreign body retrieval device from FIGS. 1 and 2;

FIG. 6 a manufacturing step in the sequential production of foreign body retrieval devices according to the invention as shown in FIGS. 1 and 2, shown before separation into the individual foreign body retrieval devices;

FIG. 7 a view of a fastening element of an endoscopic device and a foreign body retrieval device according to a second embodiment of the invention, shown before fastening of the foreign body retrieval device;

FIG. 8 a further view of the fastening element and a fastening section of the foreign body retrieval device from FIG. 7, shown during fastening of the foreign body retrieval device; and

FIG. 9 a view of a fastening element of an endoscopic device and a fastening section of a foreign body retrieval device according to a third embodiment of the invention, shown after fastening of the foreign body retrieval device.

### DETAILED DESCRIPTION OF THE INVENTION

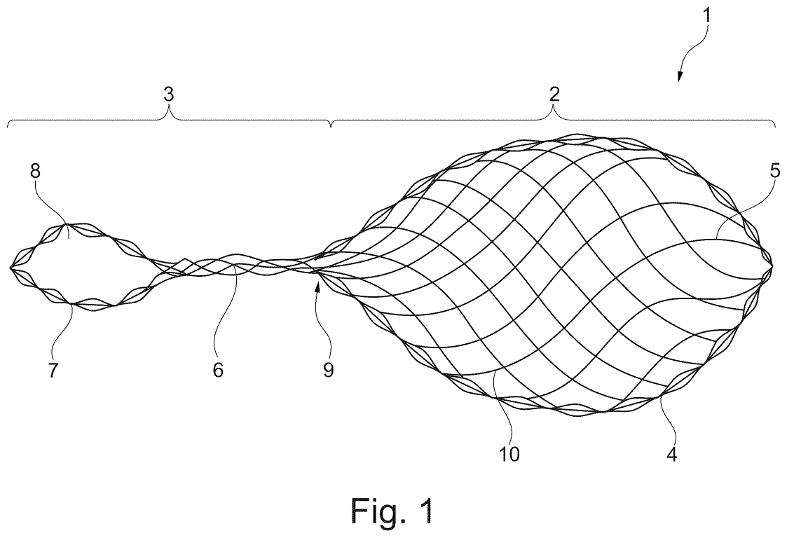

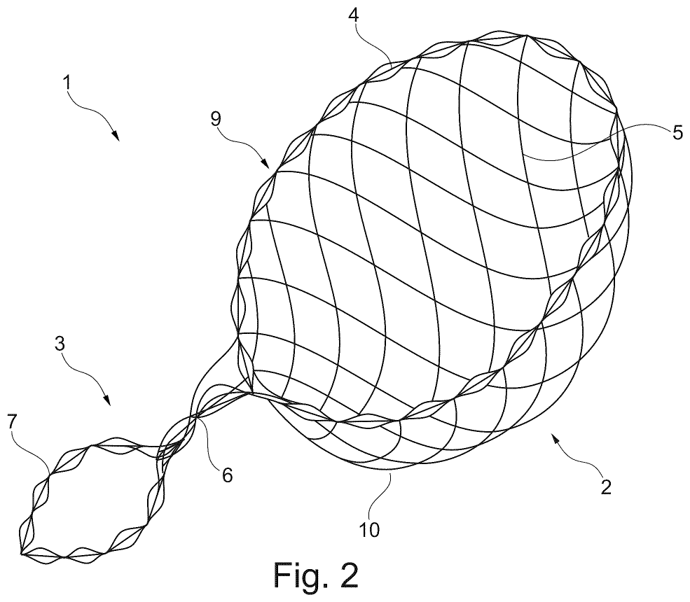

FIGS. 1 and 2 show views of a foreign body retrieval device 1, which is designed in accordance with a first embodiment of the invention. The foreign body retrieval device 1 comprises a retrieval section 2 and a fastening section 3, which are adjacent to each other, so that the fastening section 3 is designed to be immediately neighboring the retrieval section 2.

The retrieval section 2 has a support frame 4, which encircles a retrieval net 5 of the retrieval section 2. As can be seen in FIG. 2, which shows the foreign body retrieval device 1 in an unfolded state of the retrieval net 5, the retrieval net 5 is bulbous in its unfolded state.

As can be seen in FIGS. 1 and 2, the support frame 4 of the retrieval section 2 also forms a transition to the fastening section 3, which is connected to the support frame 4 by an elongated connecting part 6. The connecting part 6 connects the support frame 4 to a mounting loop 7 of the fastening section 3, wherein this mounting loop 7 is ring-shaped and defines a passage 8 through which the foreign body retrieval device 1 can be attached in a form-fitting manner.

In the present case, the foreign body retrieval device 1 is designed as a one-piece textile structure 9, which has been produced by interlinking a filament 10. The filament is designed as a monocomponent and consists in particular of a thermoplastic material, which may be polyvinyl chloride or polyethylene. The textile structure 9 is formed by knitting the filament 10, whereby the filament 10 is intertwined to form meshes and the structure 9 is thus designed as a knitted fabric. The textile structure 9 forms the retrieval section 2 and the fastening section 3 in one piece, so that the support frame 4, the retrieval net 5, the connecting part 6, and the mounting loop 7 are also realized in one piece by the textile structure 9.

As indicated in particular in FIG. 2, the structure 9 is designed with a higher mesh density in the areas forming the support frame 4, the connecting part 6, and the mounting loop 7 than in the area forming the retrieval net 5. As a result, the foreign body retrieval device 1 has a higher rigidity at the support frame 4, the connecting part 6, and the mounting loop 7 than at the retrieval net 5. In the case of the support frame 4, this ensures reliable support of the retrieval net 5 and tensioning of the retrieval net 5, whereas the higher rigidity of the connecting part 6 allows linear force transmission between the mounting loop 7 and the retrieval section 2. Due to its higher rigidity, the mounting loop 7 enables a resilient attachment of the foreign body retrieval device 1. The higher density of the meshes of the structure 9 is particularly achieved in the area of the support frame 4, the connecting part 6, and the mounting loop 7 by knitting the filament 10 in a garter stitch pattern, whereas the filament 10 is preferably knitted in a stockinette stitch pattern to form the retrieval net 5.

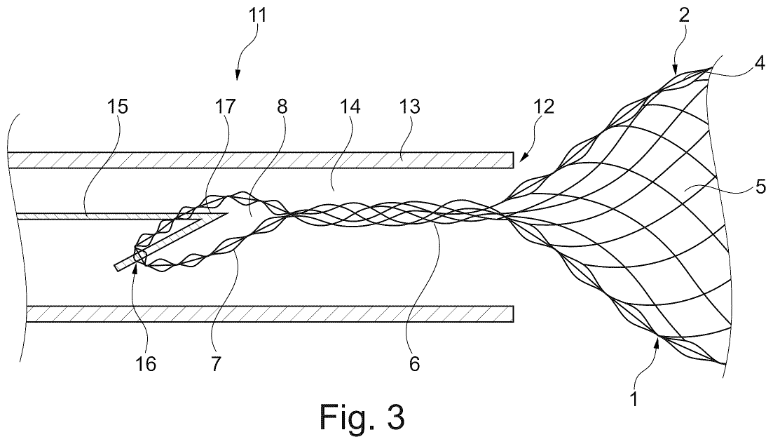

FIG. 3 shows a sectional view of a part of an endoscopic device 11, wherein this endoscopic device 11 is shown in the region of one end 12 of a flexible tube 13. This tube 13 has a lumen 14 extending in its longitudinal direction, in which an actuating element 15 extends with radial play. The actuating element 15 is an actuating wire or cable, wherein the actuating element 15 forms a fastening element 16 at its end visible in FIG. 3, to which fastening element 16 the foreign body retrieval device 1 according to the invention is fastened with its mounting loop 7. The fastening element 16 is designed as a barb-like fastening hook 17, which is inserted into the passage 8 formed by the mounting loop 7 of the foreign body retrieval device 1. This achieves a form-fitting fastening of the foreign body retrieval device 1 to the actuating element 15.

At one end opposite to the attachment of the foreign body retrieval device 1, the actuating element 15 is also connected in a manner known to those skilled in the art to a handpiece (not shown here), via which a linear actuating movement can be initiated in the actuating element 15. This linear actuating movement is then also transmitted to the foreign body retrieval device 1 via the actuating element 15 due to the form-fitting connection. This allows the foreign body retrieval device 1 to be moved between a state in which it is completely retracted into the tube 13 and a state, indicated in FIG. 3, in which the foreign body retrieval device 1 is pushed out of the tube 13 with its retrieval section 2. In the retracted state, the support frame 4 of the retrieval section 2 is folded up and the retrieval net 5 is folded up accordingly, whereas in the extended state, the support frame 4 ensures that the retrieval net 5 is unfolded as shown in FIG. 2. In the unfolded state, foreign objects, such as polyps, can be picked up with the retrieval net 5 via the foreign body retrieval device. Due to the higher rigidity of the connecting part 6, a reliable transmission of the linear actuating movement from the actuating element 15 to the retrieval section 2 of the foreign body retrieval device 1 is achieved.

However, within the scope of the invention, an embodiment would also be possible in which a foreign body retrieval device designed in accordance with the invention is attached directly to a handpiece of the endoscopic device. In this case, the connecting part of the fastening section of this foreign body retrieval device would have to be designed with a corresponding length and stiffness.

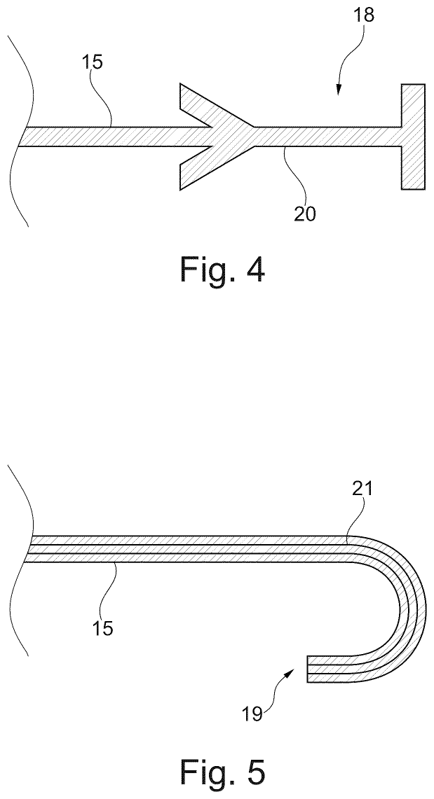

Furthermore, FIGS. 4 and 5 show alternative designs for fastening elements 18 and 19 of the actuating element 15. Here, fastening elements 18 and 19 are also designed as fastening hooks 20 and 21, but differ from fastening hook 17 in FIG. 3.

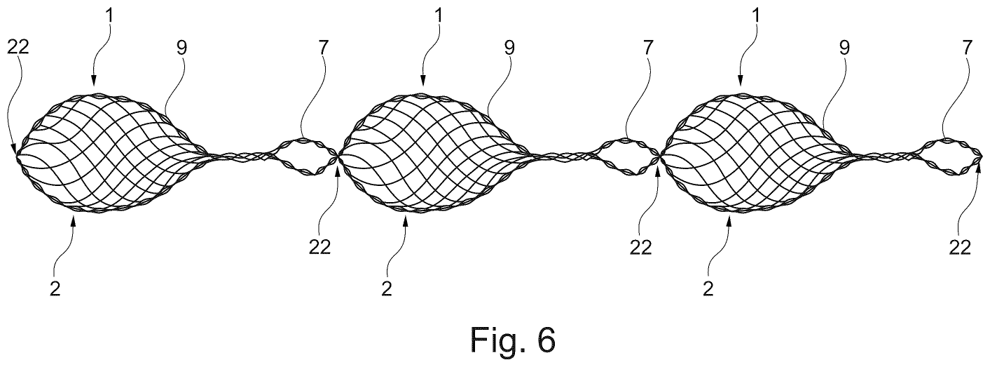

FIG. 6 shows a production stage in the production of the foreign body retrieval device 1 according to the invention. In this production process, a plurality of foreign body retrieval devices 1 are produced in one piece and in series by knitting the filament 10, whereby, when knitting the filament 10 to the mounting loop 7 of one foreign body retrieval device 1, the filament 10 is then knitted to form the retrieval section 2 of the foreign body retrieval device 1 following in the series. The foreign body retrieval devices 1 in series are then separated by separating the series of foreign body retrieval devices 1 at separation points 22. This separation is carried out by hot cutting, whereby the filament 10 is fused at the separation points 22, thereby preventing unwanted opening of the respective structure 9 of the individual foreign body retrieval device 1.

Alternatively, the separation points 22 could also be designed as predetermined breaking points, so that the foreign body retrieval devices 1 could then be separated by bending at the separation points 22.

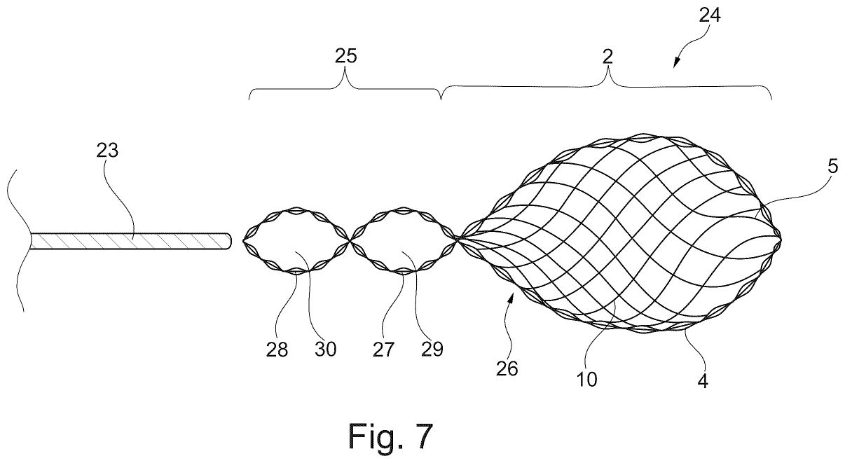

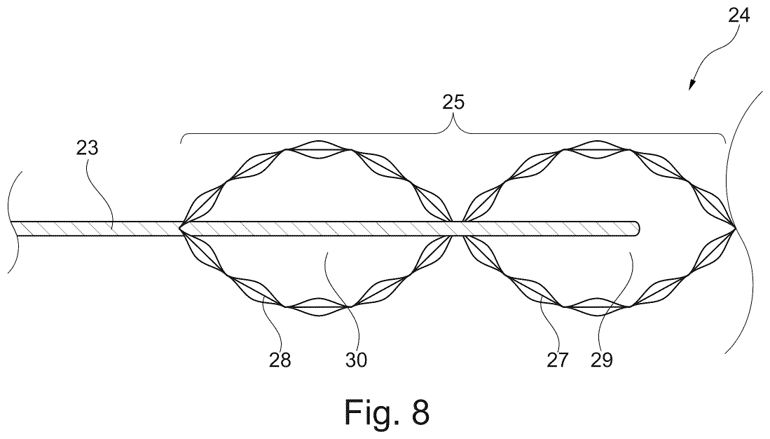

Furthermore, FIGS. 7 and 8 show views of an actuating element 23 of an endoscopic device (not shown in further detail in FIGS. 7 and 8) and a foreign body retrieval device 24. The foreign body retrieval device 24 is designed in accordance with a second embodiment of the invention and is very similar to the foreign body retrieval device 1 shown in FIGS. 1 and 2. Thus, the foreign body retrieval device 24 also comprises a retrieval section 2 and an adjacent fastening section 25, which together are designed as a one-piece textile structure 26 produced by interlinking the filament 10.

While the retrieval section 2 of the foreign body retrieval device 24 is designed to be at least substantially identical to the retrieval section 2 of the foreign body retrieval device 1 shown in FIGS. 1 and 2, the fastening section 25 has a different design compared to the embodiment shown in FIGS. 1 and 2. The fastening section 25 has two mounting loops 27 and 28, which are arranged in line one after the other, are each ring-shaped, and each define the respective passage 29 and 30. The mounting loop 27 is located between the mounting loop 28 and the adjacent retrieval section 2. Analogous to the embodiment shown in FIGS. 1 and 2, the structure 26 is designed with a higher mesh density in the areas forming the support frame 4 and the two mounting loops 27 and 28 than in the area forming the retrieval net 5, whereby the support frame 4 and also the two mounting loops 27 and 28 each have a higher rigidity compared to the retrieval net 5 of the retrieval section 2. In all other respects, the foreign body retrieval device 24 otherwise corresponds to the embodiment shown in FIGS. 1 and 2, so that reference is made to FIGS. 1 and 2 for the further configuration.

The actuating element 23, which may be an actuating wire or cable, is not equipped with a fastening element at its end shown in FIGS. 7 and 8. Instead, the actuating element 23 is threaded through the passages 29 and 30 of the mounting loops 27 and 28 for fastening to the foreign body retrieval device 24, as indicated in FIG. 8. The fastening is then achieved by press-fitting the actuating element 23 with the fastening section 25.

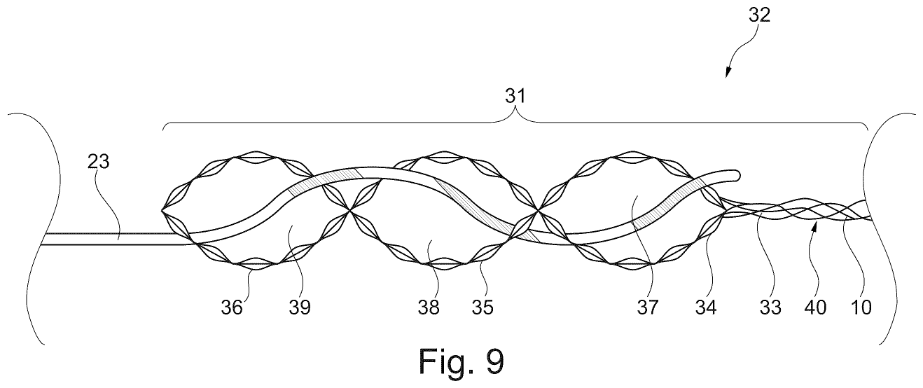

Finally, FIG. 9 shows a view of the actuating element 23 in the area of fixing to a fastening section 31 of a foreign body retrieval device 32, which is designed here in accordance with a third embodiment and of which only the fastening section 31 is shown in FIG. 9. The foreign body retrieval device 32 again is very similar to the foreign body retrieval device shown in FIGS. 1 and 2, as the foreign body retrieval device 32 has, in addition to the fastening section 31, a retrieval section 33 (not shown in FIG. 9) that corresponds at least substantially to retrieval section 2 of the foreign body retrieval device 1 shown in FIGS. 1 and 2.

In contrast to the foreign body retrieval device 1, three mounting loops 34, 35, and 36 are arranged in a row on the fastening section 31 of the foreign body retrieval device 32, in addition to a connecting part 33, which establishes the connection to the adjacent retrieval section, the loops being ring-shaped and thereby forming a passage 37, 38, and 39, respectively. The mounting loops 34 to 36 are arranged in a row, with the first mounting loop 34 located at the end of the fastening section 31 and the last mounting loop 36 located at the end of the retrieval section 32.

Similar to the embodiment shown in FIGS. 1 and 2, the fastening section 31 of the foreign body retrieval device 32 is designed in one piece together with the retrieval section (not shown) as a textile structure 40, which has been produced by interlinking the filament 10. In the areas forming the support frame of the retrieval section, the connecting part 33, and the mounting loops 34 to 36 of the fastening section 31, this structure 40 is designed with a higher mesh density than in the area forming a retrieval net of the retrieval section. This also achieves a higher rigidity of the support frame and the fastening section 31 in the foreign body retrieval device 32 compared to the retrieval net. In all other respects, the foreign body retrieval device 32 corresponds to the embodiment shown in FIGS. 1 and 2, so that reference is made to FIGS. 1 and 2 for the further configuration.

In FIG. 9, the actuating element 23 of the endoscopic device is now attached to the foreign body retrieval device 32 by threading the end of the actuating element 23 through the passages 37 to 39 of the mounting loops 34 to 36 and then press-fitting it to the fastening section 31.

The foreign body retrieval devices 24 and 32 shown in FIGS. 7 to 9 can also be manufactured in a manner analogous to that described in FIG. 6, in that several foreign body retrieval devices 24 or 32 are manufactured in one piece and in series by knitting filaments. The only difference in the manufacturing process is the production of the respective fastening section 25 or 31, since in this case several mounting loops 27 and 28 or 34 to 36 must be formed before the retrieval section of the foreign body retrieval device 24 or 32 following in series is then manufactured in one piece.

The embodiments according to the invention enable the creation of a foreign body retrieval device that is characterized by low manufacturing costs and can also be reliably attached to an endoscopic device.

While specific embodiments of the invention have been shown and described in detail to illustrate the inventive principles, it will be understood that the invention may be embodied otherwise without departing from such principles.

I claim:

1. A foreign body retrieval device for an endoscopic device, comprising a retrieval section, that forms a retrieval net, and a fastening section adjacent thereto, which is provided for fastening the foreign body retrieval device in the endoscopic device, wherein at least the retrieval net of the retrieval section and the fastening section are in one piece as a textile structure which is produced by interlinking at least one filament, wherein at the fastening section the structure forms at least one mounting loop, at which the foreign body retrieval device is to be fastened in the endoscopic device.

2. The foreign body retrieval device according to claim 1, wherein in the retrieval section the retrieval net is surrounded by a support frame.

3. The foreign body retrieval device according to claim 2, wherein the support frame is designed in one piece together with the retrieval net and the fastening section as the textile structure and has a higher rigidity than the retrieval net.

4. The foreign body retrieval device according to claim 3, wherein the higher rigidity of the support frame compared to the retrieval net is achieved by forming the support frame with a higher density compared to the retrieval net.

5. The foreign body retrieval device according to claim 1, wherein the fastening section has a higher rigidity than the retrieval net.

6. The foreign body retrieval device according to claim 5, wherein the higher rigidity of the fastening section is achieved in comparison to the retrieval net by the fastening section being formed with a higher density in comparison to the retrieval net.

7. The foreign body retrieval device according to claim 1, wherein the fastening section is coupled to the retrieval section via an elongated connecting part, which connects the retrieval section to the at least one mounting loop.

8. The foreign body retrieval device according to claim 1, wherein the at least one filament is designed as a monocomponent, preferably made of a thermoplastic material, particularly preferably made of polyvinyl chloride or polyethylene.

9. The foreign body retrieval device according to claim 1, wherein the textile structure forms exactly one mounting loop at the fastening section.

10. The foreign body retrieval device according to claim 1, wherein the textile structure forms a plurality of mounting loops, preferably exactly two or exactly three mounting loops.

11. An endoscopic device with a flexible tube that has at least one lumen running in its longitudinal direction, wherein with radial play through the lumen there runs an actuating element to which a foreign body retrieval device according to claim 1 is connected.

12. A method for manufacturing a foreign body retrieval device for an endoscopic device, wherein a retrieval section forming a retrieval net and a fastening section being adjacent thereto and provided for fastening the foreign body retrieval device in the endoscopic device are produced, and wherein at least the retrieval net of the retrieval section and the fastening section are jointly designed as a one-piece textile structure which is formed by interlinking at least one filament, wherein at least one mounting loop is formed by the structure at the fastening section, at which mounting loop the foreign body retrieval device is to be fastened in the endoscopic device.

13. The method according to claim 12, wherein a plurality of such foreign body retrieval devices are produced sequentially in one piece as a textile structure by interlinking the at least one filament, wherein the foreign body retrieval devices are then separated from one another at separation points.

14. The method according to claim 13, wherein the separation is carried out by means of hot cutting.
